(19) United States
(12) Patent Application Publication
(10) Pub. No.: US 2006/0025790 A1
(43) Pub. Date: Feb. 2, 2006

(54) Operation element, operation set and method for use thereof

(75) Inventors: Erwin de Winter, Wilrijk (BE); Cornelis Antonius F. Tulleken, Utrecht (NL); Hendricus Jacobus Mansvelt Beck, Bilthoven (NL); Jochem-Paul Bremmer, Utrecht (NL)

Correspondence Address: BANNER & WITCOFF, LTD., 28 STATE STREET, 28th FLOOR, BOSTON, MA, 02109-9601, US

(73) Assignee: UMC Utrecht Holding B.V., Utrecht (NL)

(21) Appl. No.: 11/021,553

(22) Filed: Dec. 23, 2004

Related U.S. Application Data
(60) Provisional application No. 60/585,998, filed on Jul. 7, 2004.

(30) Foreign Application Priority Data
Dec. 23, 2003 (EP) 03079163.6

(51) Int. Cl. A61B 17/08

(52) U.S. Cl. 606/153

(57) ABSTRACT

This invention provides an operation element for use in by-pass surgery, comprising a ring shaped element having windings, preferably at least one-and-a-half winding, at least partly of wire material in close proximity, provided with at least one sharpened end.

### RELATED APPLICATION

This application claims priority from U.S. Provisional Patent Application No. 60/585,998, filed on Jul. 7, 2004, hereby incorporated by reference in its entirety for all purposes.

### BACKGROUND OF THE INVENTION

The invention relates to an operation element. The invention especially relates to an operation element for use in vascular related surgery such as by-pass surgery for connecting a shunt to a blood vessel such as a vein, artery, vessel or the like.

In by-pass surgery an opening has to be provided in a vessel to which a shunt has to be connected for forming a by-pass to for example a blood clogging, a blockage of said vessel or the like. If the flow of blood through the relevant vessel can not temporarily be stopped, the shunt has to be connected while the blood still flows through said vessel, such as non-occlusive anastomosis. Such is for example often necessary in by-pass surgery in or near the brain, which is a form of surgery on a very small scale.

In brain surgery it is commonly known to use a part of a donor vessel, for example taken from a patient's leg, as a shunt. This shunt is sewn to the outside of a vessel to which it is to be connected, by suture. The surgeon has to suture the free edge of the shunt to said vessel through the wall of said vessel. This is commonly to be performed deep within the skull, leaving the surgeon very little space to operate, whereas the view on the operating surface will be limited by body tissue, instruments and the surgeon. After the shunt has been sutured to said outside wall of said vessel, the wall part of the vessel within the free end of said shunt is removed, preferably using a laser. The shunt is then flushed for a short period, in order to remove any possible debris, after which the shunt is clamped shut and connected with the opposite end to an other part of the same vessel or to a different vessel, in a similar manner. Such technique is the ELANA (Excimer Laser Assisted Non-occlusive Anastomosis) operation technique, which has been developed and optimised by Prof. Dr. C. A. F. Tulleken, University Medical Centre of Utrecht, The Netherlands and the Annemarie Tulleken Foundation, The Netherlands.

Due to the suturing and the small and restricted operating space this method is difficult and time consuming, whereas it may be prone to irregularities.

U.S. Pat. No. 6,352,543 further discloses an operation set comprising a magnetic set of rings, one of which is inserted into a vessel to which a shunt has to be connected and the other outside, holding said first ring through the vessel wall. An opening as large as said ring is made in the vessel wall for introducing it. Of such set the ring within the vessel could get free of the other ring. Moreover, such set is unsuitable for non-occlusive surgery.

In a first aspect of the present invention there is provided an operation element to be used in by-pass surgery as for example described here above, which prevents or alleviates at least some of the problems of at least one of the known methods.

In an other aspect of the invention there is provided an operation element, which is easy to use, especially in positioning thereof and in connecting a shunt.

In a still further aspect of the present invention there is provided an operation element which can simplify the operating procedure, which can provide for a secure connection and is minimal invasive.

In a further aspect of the invention there is provided for an operation element suitable for use with non-occlusive anastomosis, especially for but not limited to carnal or brain surgery.

### SUMMARY OF THE INVENTION

In a first embodiment there is provided for an operation element comprising a first part for positioning outside a blood vessel and at least a second part for positioning inside said blood vessel, either within a wall of said vessel or on an inside surface of said wall. Said second part is connected to said first part and has a free end for extending into and/or through said wall, preferably provided with puncturing means for puncturing said wall of said blood vessel, wherein during use said first and second part enclose a wall part of said blood vessel.

In use the first part is kept outside the vessel to which the shunt is to be connected. The second part is introduced into or through said wall, such that it will substantially flatten a part of said wall between at least part of the first and second parts of said operation element.

In another embodiment of the invention an operation element is provided with a ring shaped element, at least partly made of wire and having a sharpened end. The ring shaped element has windings, at least partly in close proximity.

An end is preferably provided with sharpened end, designed such that it can be pricked through the wall of a blood vessel without excessive force. In an alternative embodiment said wall can be punctured with a suitable instrument, such as a needle, such that said end, which may then be blunt, can be introduced through said puncture.

The operation element can then be moved, in particular be rotated and/or translated such that at least part of said second part can be moved into said vessel, in or under said wall through which said sharpened end has been moved. Since said first and second parts are at least partly in close proximity said wall will be clamped between said windings. This results in a close fit, enabling easy and accurate positioning of the operation element on a wall of a vessel, using simple instruments such as a pincer or pliers. Part of the operation element can be kept on the outside of the wall of said vessel, such that this provides a surface for connecting a shunt.

Since at least part of said operation element will be positioned in or under said wall, the first part can be used as a working surface, similar to an anvil, which will be stable and allow the surgeon to work on.

In a further embodiment of the invention, the operation element, especially a ring shaped element thereof has at least 1.5 windings, more preferably between 1.5 and 2 windings, more specifically between 2 and 4 windings. This means that approximately one winding can be brought under the wall inside a vessel whereas also approximately one winding or more windings can be kept on the outside of said wall. The ring shaped element is then simple in construction, easy to manufacture and will provide for approximately a full circle on top of and under said wall.

In a still further embodiment the ring shaped element has a first and second winding, connected to each other by a connecting part. The first and second winding are both substantially flat and lie parallel to each other, the connecting part being bent. This results in a ring shaped element that in itself is flat. During use the first winding can lie under said wall, inside said vessel, the second winding lying on said wall, outside said vessel. The connecting part will then stick through said wall. Due to the flat construction the wall part of the vessel to which the operation element is connected will be flattened, providing for a substantially flat wall part within a central opening of said ring shaped element.

A ring shaped element of an operation element according to the present invention may be substantially circular or oval.

In a particularly embodiment the sharpened end of said ring shaped element is bent relative to the direction of winding. This has the advantage that when positioning the ring shaped element by rotating thereof in the direction of winding after penetrating said wall with said sharpened end for the first time for bringing this into said vessel, the sharpened end will not be prone to be forced into or through the wall of said vessel again. Preferably said end is bent backward relative to the direction of winding, more preferably lying in a imaginary cylindrical surface defined by the windings.

The ring shaped element can be made of wire. The wire preferably has a substantially circular cross section with a diameter of less than 2 mm, preferably between 0.1 and 2 mm and more preferably approximately 0.25 mm and can be chosen depending on the intended use, that is type and place of surgery. In an alternative embodiment wire the cross section of similar size, but with a flattened portion, such that of part of windings abutting each other are flatted for increasing the contact surface and therefore the clamping force exerted on the wall of a vessel. In a still further alternative embodiment the ring shaped element is made from a tube which is slid in the wall for forming a spiralling incision.

In a further alternative embodiment the ring shaped element can be connected to or part of a tube shaped element, such that a winding of said ring shaped element can be brought within a vessel, as described before, whereas said tube shaped element will be kept on the outside of said vessel, to which element a shunt can be connected.

The invention further relates to an operation set, comprising an operation element as described and a connecting element for connecting to said operation element. Such connecting element will enable easy connection of a shunt to said operation element, further facilitating easy use.

The connecting element preferably comprises hook shaped elements, which can hook around the ring shaped element, at least on the outside of said vessel. This enables suture free connection. Preferably a locking element can be forced over said hook shaped elements, for locking these in position around said ring shaped element.

In a further embodiment a ring is provided with a central opening, which can be enclosed within a folded back free end of a shunt to be connected to said operation element. This ring will provide stability to a folded edge formed by said folded back free end. The ring can preferably either fit inside a central opening of said ring shaped element or fit on top of said ring shaped element. A clamping element may be provided for clamping said shunt onto said ring shaped element.

In an alternative embodiment said first part comprises a curved segment including an angle of more than 180 degrees, having opposite first and second ends, a second part connected to at least one of said first and second ends by a curved intermediate part, wherein said at least one second part at least partly extends in a plane substantially parallel to a plane defined by at least said first and second ends of said first part and at least one further part of said first part between said first and second ends, which second part has a free end opposite said intermediate part which is spaced apart from said first and second ends. This clip like operation element can be positioned on a wall of a bodily part by inserting into or through the wall of a bodily element the second part, substantially by translation.

The present invention further relates to a set of an operation set according to the present invention and a shunt. In the present invention a shunt can at least be a natural shunt such as part of a blood vessel, or can be artificial. The shunt preferably has an inner cross section substantially equal to the cross section of the ring shaped element, especially of a central opening thereof.

The present invention further relates to a shunt for use with an operation element or set according to the present invention, which shunt has a first end folded back and outward over a ring.

The present invention furthermore relates to a method for connecting a shunt to a blood vessel.

With a method according to the present invention a surgeon can relatively easy prepare a bodily part such as a vessel, urethra, bladder or the like, comprising a bodily fluid, for connecting a shunt, even in non-occlusive anastomosis. Since the ring shaped element can be position with a limited set of relatively simple tools, the surgeon has relatively much freedom to operate. He will not be hampered by for example suturing devices and the like. The ring shaped element can be placed suture free. The surgeon can freely decide how to connect a shunt to said operation element.

In a method according to the present invention a ring shaped element can be used having a first and second winding, connected by a connecting part, whereby the first winding is brought under a wall inside a vessel whereas the second winding is kept on the outside of said wall. The wall is clamped between the first and second winding, leaving only the connecting part traversing said wall. The ring shaped element can to that end be rotated around a longitudinal axis.

In such embodiment it may be preferable that a ring shaped element is used having a sharpened end bent backward relative to the direction of winding, such that the ring shaped element is first moved in a first direction, against the winding direction, for bringing the end through said wall, after which the ring shaped element is rotated in the opposite direction, the winding direction, for bringing the first winding under said wall, within said vessel. To that end the ring shaped element is rotated for example over an angle of more than 270°, more specifically over an angle of approximately between 320 and 360°.

In a specific embodiment a method according to the present invention is further characterised by the features of claim 23.

This method provides for a stable end of the shunt to be connected to the ring shaped element, compatible to said ring shaped element is dimensions and form. This enables relatively easy fluid tight connection of the ring shaped element and the shunt.

In a still further embodiment the wall part of said bodily part extending within a central opening of the ring shaped element, which wall part may be substantially flat, is sucked outward a little, into said central opening, before removal thereof. This leads to an opening in the wall within said central opening with a more desirable shape and dimensions, having smoother edges enhancing uninhibited blood flow through the shunt and blood vessel.

Since the first and second parts of operation elements according to the invention are interconnected the second part can not accidentally be detached within said vessel. Moreover, the operation element can be used in both occlusive and non-occlusive operations.

In the sub claims advantageous embodiments are given of the present invention. For a better understanding of the present invention embodiments thereof will be described hereafter, with reference to the drawings.

### BRIEF DESCRIPTION OF THE DRAWINGS

FIG. 1A-D show on an enlarged scale an operation element according to the present invention from various angles;

FIG. 1E shows an alternative embodiment of an operation element according to the invention;

FIG. 1F shows in perspective view an embodiment of an operation element according to the invention;

FIGS. 1G and H show in cross section along to the line ID-ID two embodiments of the attachment of a shunt to a bodily part, such as a vessel, using an operation element according to the invention;

FIG. 1I shows in partial cross section an embodiment of a connection according to FIG. 1F, in a further alternative embodiment;

FIG. 2A-G show different steps in the attachment of a shunt to a vessel according to the present invention;

FIG. 2H shows in cross section part of a connection made according to FIG. 2A-2G;

FIG. 3A-D show in elevated view, partly broken away, top view and two perpendicular side views respectively an alternative embodiment of a operation element shaped element according to the present invention;

FIG. 4 shows in a cross section according to FIG. 3C an operation set on a blood vessel, in further enlarged scale;

FIG. 5 shows in perspective view, partly broken away, a plug and receiver of an embodiment according to FIGS. 3 and 4, as well as the plug combined with a shunt;

FIG. 6A-D show in elevated view, top view and two perpendicular side views respectively a further embodiment of an operation element according to the invention;

FIG. 7 shows in cross section in side view a bodily part, especially a blood vessel and a shunt connected thereto using an operation set with an operation element according to FIG. 6;

FIG. 8 shows in perspective view a further embodiment of an operation element according to the present invention; and

FIG. 9 shows a still further embodiment of an operation element according to the present invention.

### DETAILED DESCRIPTION OF THE PREFERRED EMBODIMENTS

In the following description identical or corresponding components will be referred to with the same or corresponding reference signs. The embodiments shown are only shown by way of example and should by no means be understood as limiting the scope of protection in any way. Especially combinations of features of the various embodiments are considered falling under the present invention and disclosed hereafter. Any reference to sizes and dimensions, shapes, materials or known apparatus used in the description should be considered as examples only, unless specifically stated otherwise.

As of 1993 the ELANA operation technique has become available, allowing non-occlusive bypass surgery, especially in the brain. In this technique an excimer laser is used for opening the wall of a blood vessel after connecting a shunt to said wall. To this end a metal ring is sutured to said wall, to which ring then an end of said shunt is connected by further suturing. The end of said laser is then introduced into said shunt, facing the wall within said ring, which wall part is then removed by said laser. This operation technique is very successful but has the main disadvantage that connecting said ring to said wall and connecting said shunt to said ring, both by suturing is cumbersome, especially with small sizes and in restricted operating areas such as in brain surgery. The ELANA technique as discussed in Tulleken C A, Verdaasdonk R M, Beck R J, et al: “The modified excimer laser-assisted high-flow bypass operation,” Surg Neurol. 46:424-429, 1996; and Tulleken C A, Verdaasdonk R M, Mansvelt Beck H J “Nonocclusive excimer laser-assisted end-to-side anastomosis,” Ann. Thorac. Surg. 63:S138-S142, 1997 is considered to be enclosed herein by reference.

The present invention is related to elements, sets and methods for any type of surgery in which a connection is made between a bodily part of a human or animal and a shunt such as an artery, vessel or the like for containing a bodily fluid. A bodily fluid can for example be blood, plasma, urine or excrement. Embodiments will be shown for examples of bodily parts and shunts. However, all elements, sets or methods can be used in any other type of connection as indicated.

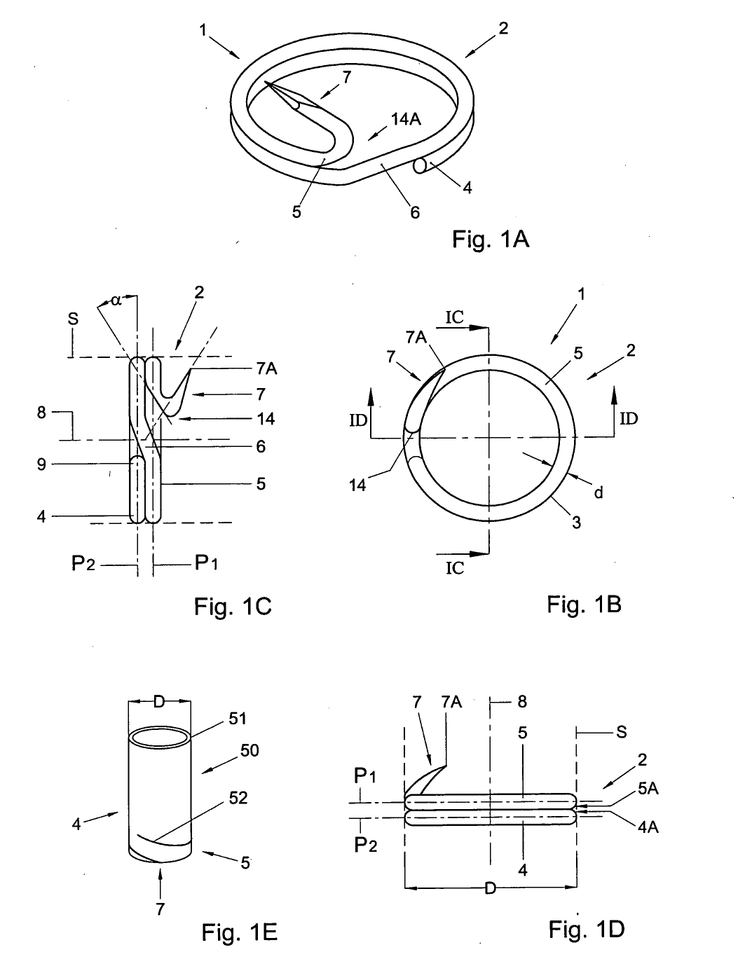

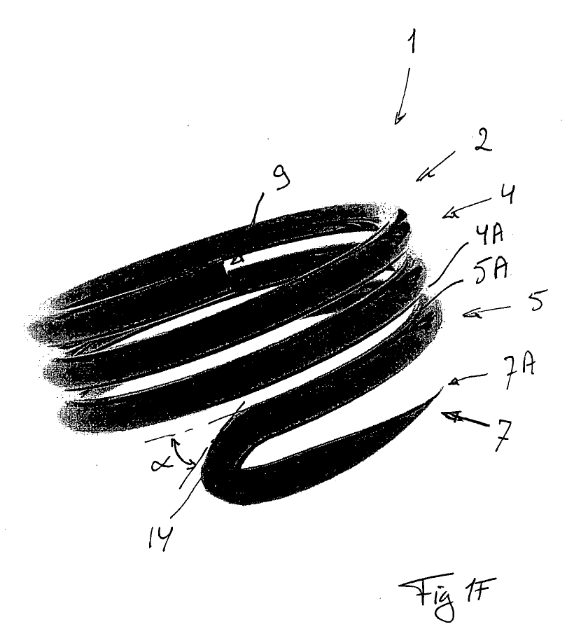

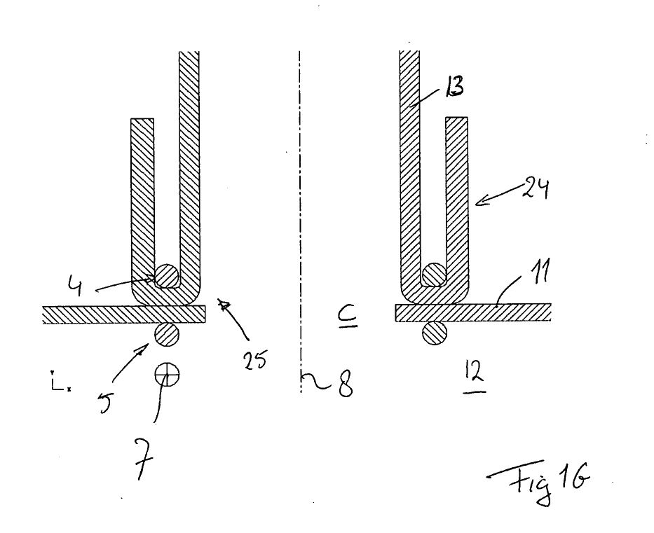

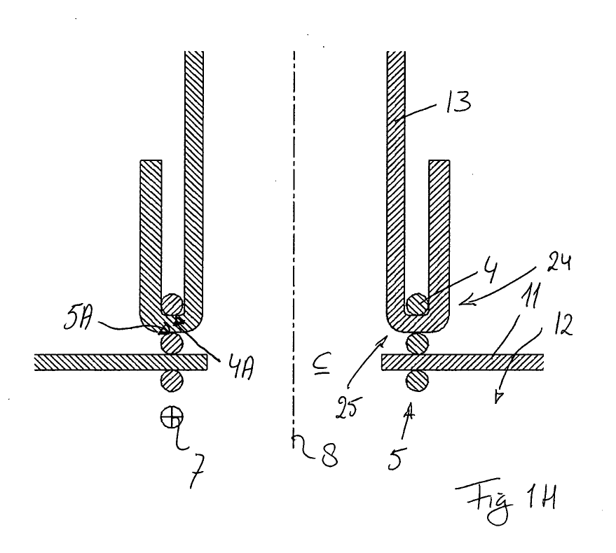

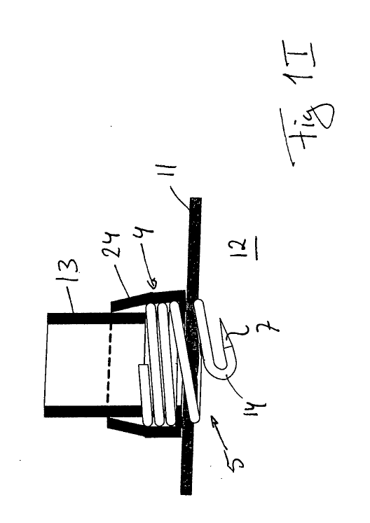

FIG. 1A shows in perspective view an operation element 1 according to the present invention, whereas FIG. 1B shows the operation element from above. FIGS. 1C and 1D show the operation element in perpendicular side views according to the arrows IC-IC and ID-ID as shown in FIG. 1B. This operation element 1 comprises or is constituted by a ring shaped element 2. This ring shaped element is made of metal wire 3 having for example a circular cross section with a diameter d of several micrometer, whereas the ring shaped element 1 as such has a diameter D of a few millimetre. The sizes d and D are dependent on the operation to be performed and especially the bodily part such as a blood vessel onto which said operation element 1 is to be used. By way of example only, the diameter d of the wire can for example be between 0 and 2 mm, especially between 0.1 and 2 mm and the diameter D of the ring shaped element 1 can for example be between 1 and 40 mm, especially 1 and 25 mm. For brain surgery for example a ring having a diameter D of approximately 2.8 mm and a diameter d of the wire of approximately 0.25 mm can be used. The wire is made of a bio compatible material, FDA approved, and can for example be made of platinum or other metal, plastic or the like.

The ring shaped element 2 comprises more than one winding, preferably at least one-and-a-half winding. In the embodiments shown the ring shaped element 2 comprises approximately two windings. More specifically the ring shaped element 2 comprises a first part 4 and a second part 5, both substantially flat, lying approximately parallel to each other and comprising a little less than a full circle. Each part 4, 5 encloses for example an angle of 320° to 340°. The first part 4 is connected to the second part 5 by a connecting intermediate part 6 formed by a substantially elongated S-shaped wire element.

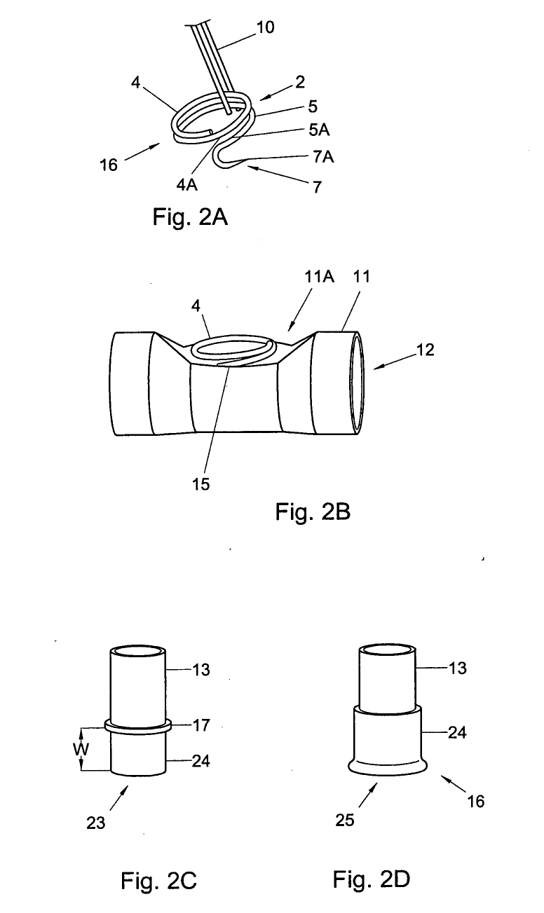

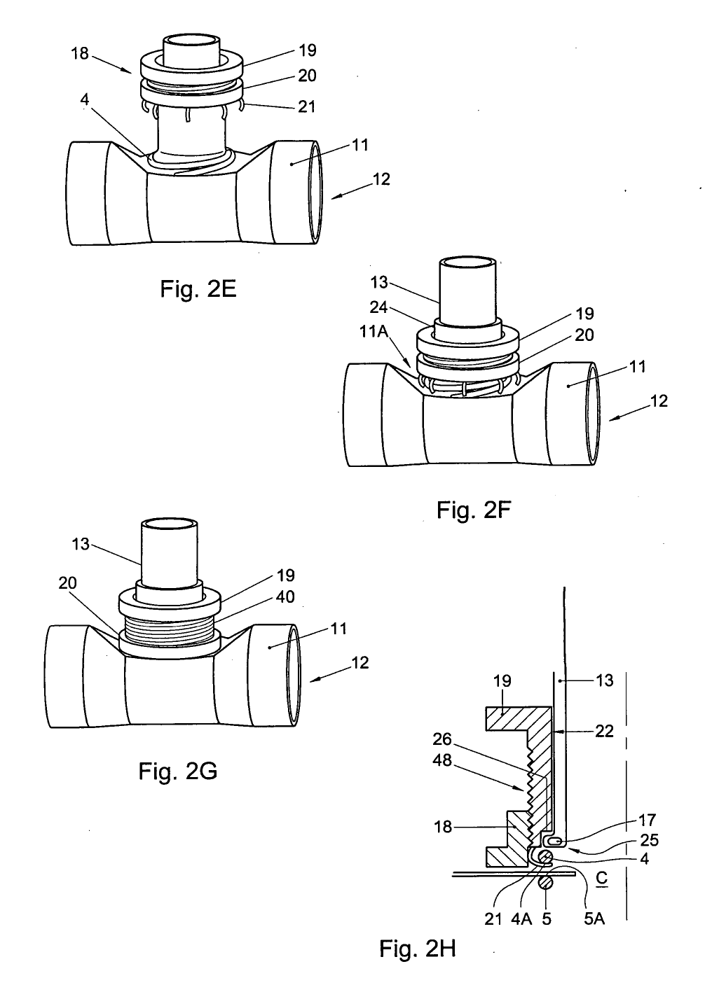

The second part 5 has a free end 7, which has a sharpened point 7A, such that it can relatively easy penetrate the wall 11 of a blood vessel 12 (FIG. 2) or another bodily part. The free end 7 is bent back and away from the first part 4. The ring shaped element 2 has a central axis 8 extending perpendicular to the planes P1 and P2 defined by the first 4 and second part 5 respectively, such that the said parts 4, 5 define a substantially cylindrical surface S on the outside of said ring shaped element 2. The said free end 7 is preferably bent such that it lies parallel to or in said outer surface S but it may also cross said surface S. The free end 7 encloses an angle □ with said planes P1, P2, preferably an acute angle, more specifically an angle of between 10 and 60°. This has the advantage that if the element 2 is rotated with said bend 7A forward, it will be forced under the wall of the bodily part more easily, as will be discussed later. The free end 9 of the first part 4 is preferably blunt.

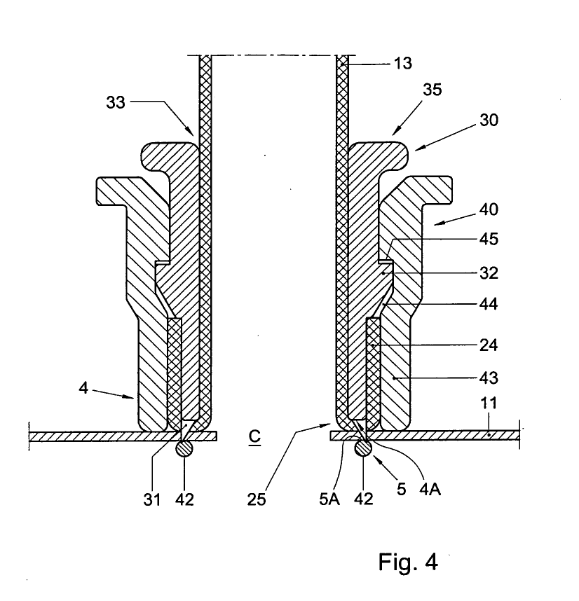

The material used for the ring shaped element 2 is preferably resilient, such that said first and second part 4, 5 are clamped onto each other, with a relatively high modulus of elasticity. Due to the shape of the cross section of the wire the contact surface between said two parts 4, 5 is preferably very small, more preferably a line-contact, such that a relatively high clamping force is obtained. The purpose thereof will be explained hereafter, especially with reference to FIGS. 2 and 4.

In operation the operation element will be connected to a blood vessel or other bodily part containing a bodily fluid as follows. The ring shaped element 2 will be picked up by an operation tool, for example a pincer 10, for example substantially parallel to the axis 8, distanced from the sharp free end 7. Said sharp free end 7 is forced through the wall 11 of a blood vessel 12 at a position where a shunt 13 such as a donor blood vessel or artificial vessel has to be connected. The ring shaped element is then forced into the direction of said wall 11 further, until the bent 14 between the free end 7 and the second part 5 reaches said wall 11. During this first step the ring shaped element 2 is rotated slightly around the axis 8 and/or translated in a first direction, in the embodiment shown counter-clockwise.

When the said bent 14 is introduced in said wall 11 the ring shaped element 2 will be rotated around said axis 8 in the opposite second direction, in the embodiment shown clockwise, such that said wall 11 is forced between the first and second part 4, 5. Due to the sloping part 14B of the bent 14, including said angle α, the second part 5 is forced under said wall 11. The ring shaped element 2 will preferably be rotated over such an angle that the connecting part 6 will be next to the opening 15 in the wall 11 through which the free end 7 is introduced or may be at least partly be introduced into said opening 15. This rotation leads to the effect that the second part 5 will be introduced into said blood vessel 12, lying against the “inside” of said wall 11, whereas the first part 4 will be maintained outside said vessel 12, lying against the “outside” of said wall 11. The wall 11 itself will thus be clamped between said first 4 and second part 5, especially between facing surfaces 4A, 5A thereof. Due to the free end 7 being bent backward the blunt side 14A of the bent 14 will be directed forward seen in the direction of rotation (clockwise) for introducing the second part 5 into said vessel 12. This prevents the free end 7 from puncturing other parts of the wall 11.

Due to this construction and method, the ring shaped element 2 will be firmly held in position and form a firm basis for attachment of the shunt 13. No suturing is necessary for positioning said ring shaped element 1 and placing the ring shaped element 1 is a relatively simple procedure.

A shunt 13 can be connected to said ring shaped element 2, especially to the first part 4 thereof in a known manner, using suturing and the basics of the ELANA technique or any other suitable technique. However, preferably the ring shaped element forms part of a set 16, further comprising a second ring 17 and a connecting element 18. The second ring 17 preferably has a diameter approximately equal to the diameter D of the ring shaped element 2, especially the first part 4 thereof and/or to the outer diameter of the shunt 13 to be connected. Approximately has to be understood such that some stretching or compression of the shunt may be allowed for fitting around said ring, whereas the second ring 17 can fit on, in or around said first part 4. The connecting element 18 comprises an upper ring 19 and a lower ring 20. The lower end of the upper ring 19 is provided with a number of hook shaped elements 21 of a resilient or pivotable nature. The hook shaped elements 21 are placed and dimensioned such that in a first position (as shown in FIG. 2E) that they can be slide over the outer periphery of the first part 4 of the ring shaped element 2, as will be discussed hereafter. The lower ring 20 can be forced down along the upper ring 19, over the hook shaped elements 21, forcing the hook shaped element 21 inwards towards a second position (shown in FIGS. 2F and G) The lower ring 19 can then be fixed in said position, for holding the hook shaped elements in said second position. The upper ring 19 has an inward facing flange 26 at its lower end, which flange 26 is ring shaped and has a central opening 22 corresponding approximately to the inner diameter of the second ring 17, such that said second ring 17 can not pass said flange and thus can not pass said connecting element through the upper and lower rings 19, 20.

A set 16 can be used as follows.

The ring shaped element 2 (FIG. 2A) is positioned on the wall 11 of a vessel 12 as described here above (FIG. 2B) Due to placement of the ring shaped element 2 the relevant wall part 11A of the vessel 12 is flattened. The second ring 17 is placed over a free end 23 of a shunt 13, at a distance W from said free end (FIG. 2C) forming a skirt 24 The skirt 24 is then folded back over said second ring 17 (FIG. 2D) enclosing the second ring 17. Thus a folded end 25 is formed, supported by the second ring 17. Said folded end 25 is position against the first part 4 of the ring shaped element 2. The connecting element 18 is moved over the shunt 13 towards the ring shaped element 2, the lower ring 20 facing said ring shaped element 2 (FIG. 2E) The connecting element 18 is forced down against the ring shaped element, such that the hook shaped elements 21 are next to the first part 4 of the ring shaped element 2, the upper ring 19 resting on the lower ring 20 (FIG. 2F) Finally the lower ring 20 is forced down along the upper ring 19, for example by intermeshing screw threads 48 or a push fit or the like, such that the hook shaped element 21 are forced at least partly under the first part 4, locking the connecting element 18 in place. At the same time the flange 26 is forced against the second ring 17 with the wall of the shunt 13 in between, pressing the folded end 25 of the shunt in a fluid, especially blood tight connection. In this position the upper and lower rings 19, 20 are locked and the connection is made, as shown in FIG. 2H.

After making said connection the ELANA technique is used for removing the wall part within said ring shaped element 2, thereby forming a connecting opening C and flushing, after which the shunt is temporarily closed of by a clamp (not shown). No part of the shunt 13, second ring 17 or operation element 2 is thereby blocking the lumen L of the shunt 13. The longitudinal axis X of the lumen L encloses an angle β with the normal 8 of the planes P1, P2 which preferably is between 0° and 60°, more specifically between 0° and 45° and preferably approximately 0°, seen near the intersection of the shunt 13 and the vessel 11.

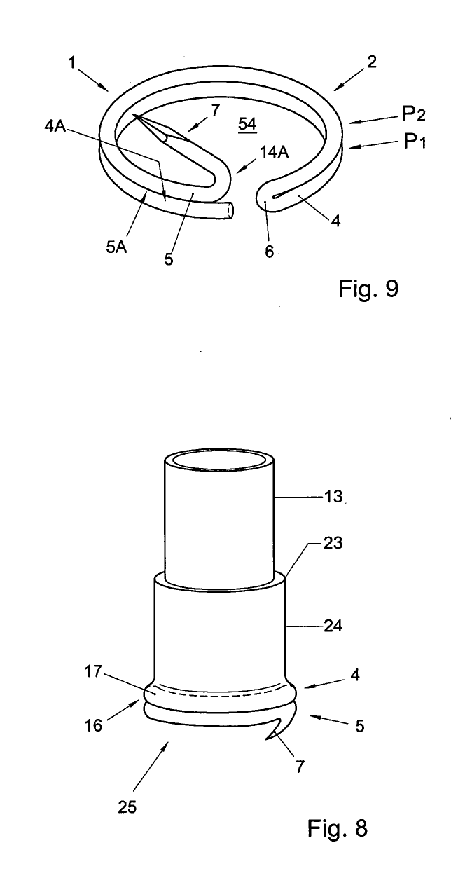

In an alternative embodiment, as shown in FIG. 8, the second ring 17 is integral with the ring shaped element 2, as the first part 4 thereof. The end of the shunt is folded over the first part 4 similar to FIGS. 2C and D, only the second part 5 extending outside. The second part 5 of the ring shaped element 2 with the connected shunt 13 is then entered into the vessel as described before. To that end an expander may be introduced into the shunt, such that the first part 4 can be engaged from the inside of the shunt 13, by pressing the expander against the wall of the shunt 13 over said first part. This will allow exerting a rotational force to said operation element and shunt.

FIG. 1E shows a further alternative, in which a tube 50 is provided, having a cylindrical outer wall 51 which has a diameter D similar to that of the first part 4 as shown in FIG. 1. A spiralling slid 52 is provided through said outer wall 51, forming the second part 5 for introducing into a wall 11 of a vessel 12 or other bodily part. The further part of the tube 50 forms the first part 4. The shunt 13 can be folded again as shown in FIGS. 2C and D. In this manner a “wire” will be obtained, forming the second part, which has a substantially rectangular cross section which may clamp the wall 11 and the shunt 13 accurately between facing surfaces 4A, 5A.

In FIG. 1F an alternative embodiment of an operation element 2 is shown, having a little more than three windings and a bent end 14 having a sharp end 7. In this embodiment the first part 4 has for example two windings and the second part 5 the rest of the windings. In this embodiment the windings, especially the facing surfaces 4A, 5A thereof to not touch. The distance between said surfaces is in the embodiment shown smaller than the diameter of the wire.

It should be noted that the indication substantially flat should in this description also be understood as enclosing a surface defined by two parallel windings or parts thereof, resulting in a slightly wound surface or helical surface.

In FIGS. 1G and 1H in cross sections two alternative embodiments of a connection made by an operation element according to the present invention are shown, comparable to FIG. 8. In these embodiments the operation element is enclosed partly in the folded end 24 of the shunt 13. In FIG. 1G approximately one winding of the first part 4 is enclosed within said folded end 24 and approximately one winding within said bodily part 12. In FIG. 1H a similar cross section is shown, in which between the folded end 25 and the outer surface of the wall 11 a further winding is enclosed.

If the graft or shunt 13 is an autologous vein, the friction of the internal side of the inversed vein with the outside of a recipient artery 12 can be quite high as the two are pressed together, making turning of the element 2 for insertion more difficult or nearly impossible. This can be overcome with the winding between the two, as shown in FIG. 1H, which also minimizes the opening of the spring and therefore the strain on the spring shaped element 2 material, so a stiffer and more prestressed spring material can be used with a higher spring tension.

The friction of the inner side of an artificial graft 13 and the outside of the wall 11 of for example a recipient artery 12 normally proves to be less of a problem, so with these the direct contact between wall 11 and graft 13 can more easily be used, as shown in FIG. 1G. This makes it easier to get a tighter fit, less prone to leakage and minimizing contact of spring with bodily fluid such as blood. Obviously, the surfaces 4A, 5A are opened wider with two layers of wall (one recipient 11, one graft 13), between them, so the material of the element 12 preferably is more flexible to enable this.

In FIG. 1I an even further embodiment of a connection is shown. Here the element 2 is shown in its entirety, whereas the shunt 13 and wall 11 are shown in cross section. In this embodiment the first part 4 comprises more than two windings, the second part 5 approximately one. The first part 4 is almost entirely embedded within the folded part 24 of the shunt 13, offering extra stiffness and easier grip for rotation. The windings of the first part 4 can be somewhat tighter than the windings of the second 5 part and/or the connection between the first 4 and second part 5. This may make insertion, especially rotation easier and the pressure distribution more even.

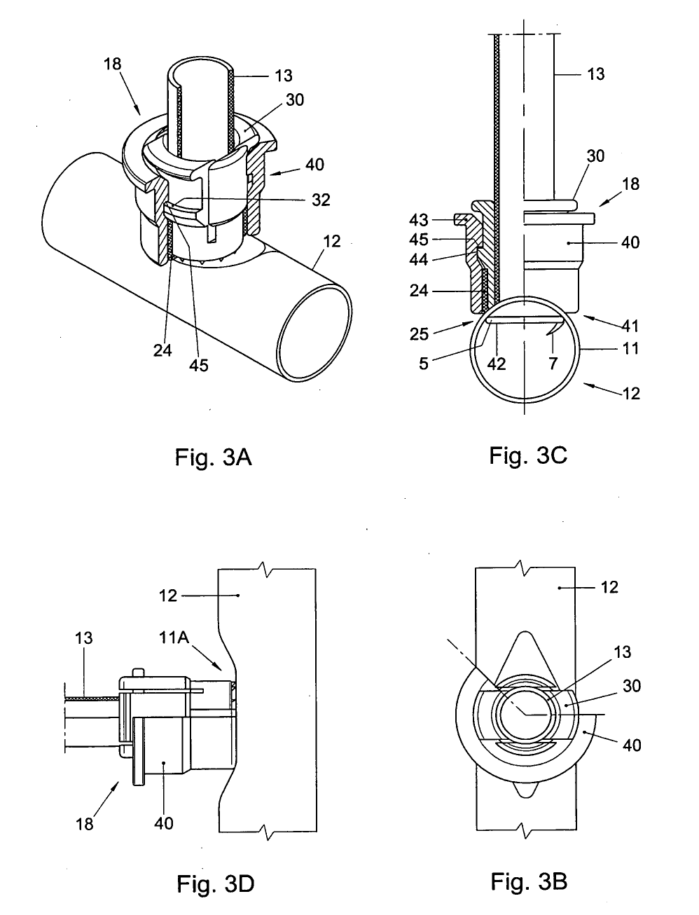

FIG. 3-5 disclose a further embodiment of an operation set according to the present invention, which will be described along with an alternative embodiment of a method for applying such.

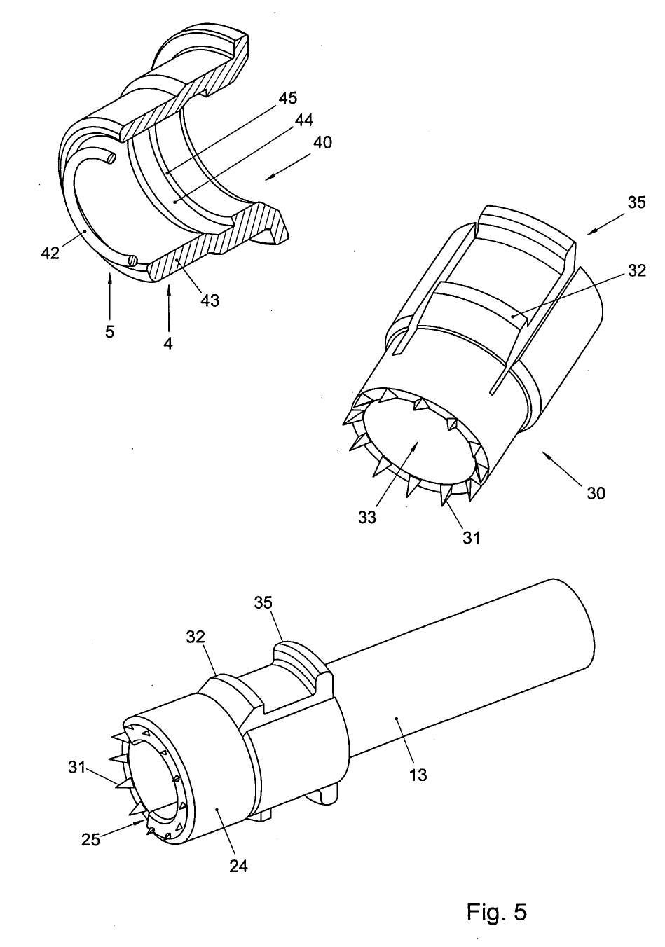

In FIG. 3A a perspective view of a second embodiment of a set 16 according to the present invention, partly cut away, positioned on a vessel 12 such as a vein or artery. In FIG. 3B a top view thereof is shown, in FIGS. 3C and 3D to side views. In FIG. 4 a cross section is given. FIG. 5 shows the shunt 13 of a set according to FIGS. 3 and 4, with the connecting element 18.

It should be emphasized that “connecting element” and “ring shaped element” are referred to a single elements, these may obviously comprise several parts or elements. The may have any shape, form or dimension as long as they are suitable as first 4 and second part 5 or complementary connecting element respectively.

An operation element 2 according to the present invention is preferably made of a resilient, flexible but considering the dimensions, relatively stiff material. Preferably the windings are wound to a spring shape in such a way and of such material and material thickness as to create a spring strong enough to exert a force on the wall 11 between the first winding which results in a pressure on the tissue between the surfaces of the spring pressing against the tissue, that is lower but not too much lower than the perfusion pressure available at the site. A pressure that is too high may damage the tissue of the wall 11 and/or shunt 13 and such element would be too difficult to introduce. Too low a pressure could result in loose fit, instability and/or leakage of fluid, such as blood, at the connection. A spring of such properties can easily be defined by a person skilled in the art of spring making. Said pressure equals the applied force per unit area and is preferably approximately 1% to 100% of the available perfusion pressure at the time the blood pressure in the recipient artery peaks during a heart cycle (=systolic blood pressure). Perfusion pressure equals the blood pressure minus the intracranial pressure. Intracranial pressure is substantially equal to the venous pressure under normal circumstances. As is obvious to persons skilled in medicine, in calculating the desired spring strength, the natural variation of blood pressure and venous pressure over longer periods and from person to person, the fluctuation of the pressures during the heart cycle and the location inside the patient's body may have to be taken into account and a reasonable safety margin be applied.

In the embodiment of FIG. 3-5 again a ring shaped element 2 is provided on a wall 11 of a vessel 12, as shown in FIG. 4. In this embodiment the ring shaped element 2 is provided in the form of a substantially cylindrical receiver 40 provided at a first, lower end 41 with a ring 42 comparable to the second part 5 of a ring shaped element according to FIG. 1, including a sharpened end 7. The cylindrical wall 43 of the receiver 40 functions as the first part 4 according to FIG. 1. On the inside of the cylindrical wall 43 a groove 44 is provided having a flat upper ridge 45. The cylindrical receiver 43 can be positioned on and connected to a wall 11 of a vessel 12 in a manner similar to the method as described with reference to FIGS. 1 and 2, by forcing the ring 42 through and under the wall 11 of the vessel 12, thereby partly flattening said wall 11.

In this embodiment in stead of the second ring 17 an inner connecting element 30 or plug is provided, having spike like elements 31 at a lower end and an outwardly extending click-ridge 32 spaced apart from said spike like elements 31. Said ridge 32 may be provided at least partly on two resilient fingers 35. The shunt 13 is introduced through a channel shaped opening 33 through said inner connecting element 31 and the free end 23 of said shunt 13 is folded back onto the outside of said inner connecting element 31 to just below said ridge 32, forming said skirt 24. The spike like elements 31 will penetrate through the folded end 25 of said shunt 13. This position is shown in FIG. 5, below, whereas the separate parts 30 and 40 are shown there above.

When the shunt 13 is prepared as described, the inner connecting part 31 with the shunt 13 is forced into the receiver 40, connected to the vessel 12 as described, having the spike like elements 31 facing forward into the direction of the wall 11. The spike like elements 31 are forced into and possibly through the wall 11 within the ring shaped element 40, preventing rotation and partly loosening said wall part 11. The click ridge 32 will be forced into said groove 44 under the ridge 45, thereby pressing the folded end 25 against the wall 11 and indirectly against the ring 42, forming a liquid tight connection and at the same time fixing the plug 30 in the receiver 40. Said ELANA technique or other suitable techniques may then be used for forming the connecting opening C.

It will be clear that a locking mechanism as disclosed in FIG. 3-5 may be used with a ring shaped element 2 according to FIG. 1 or 2, whereby the receiver may be provided with the hook shaped elements or similar connecting parts for connecting to the ring shaped element 2. Or vice versa, whereas connecting elements and techniques as described could also be used with the known ELANA technique using sutured rings.

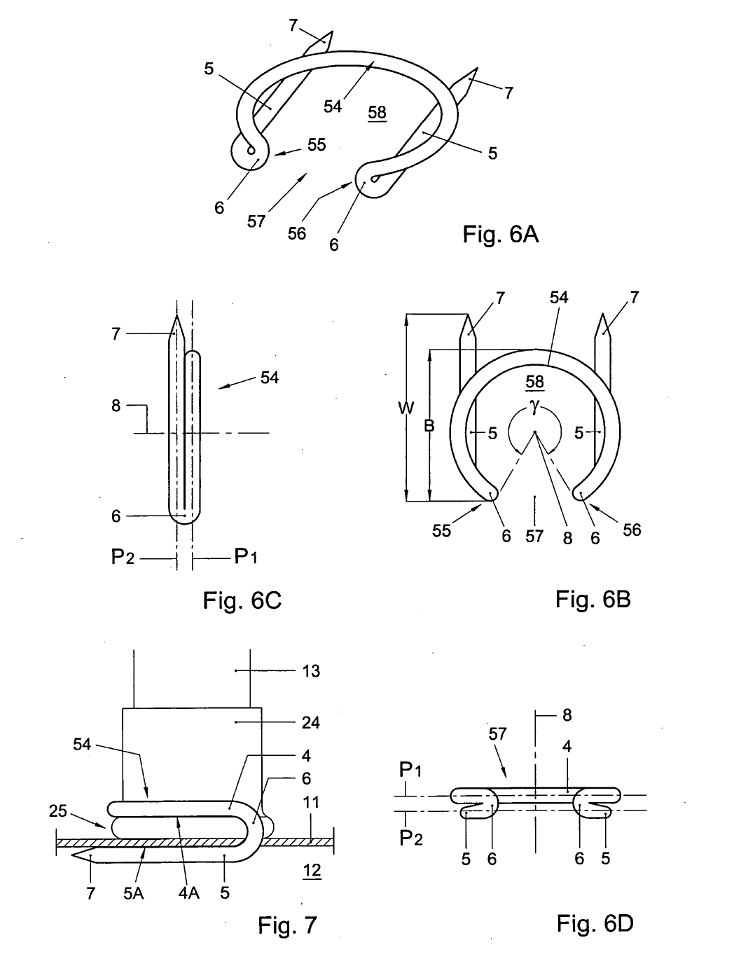

FIG. 6 shows a further alternative embodiment of an operation element 2 according to the present invention. In this embodiment a first part 4 is provided, including a curved portion 54 made of wire material, having opposite first and second ends 55, 56. To each end 55, 56 a second part 5 is connected by an intermediate part 6 which is, in the embodiment shown curved. The first part 4 extends in a plane P1, the second parts 5 in a second plane P2, substantially parallel to each other. The second parts 5 are substantially straight and parallel. They both have a forward end 7, which is shown as a sharp, puncturing end 7 but may also be blunt.

In the embodiment shown the curved portion 54 extends around a virtual axis 8, including in the first plane P1 an angle γ of more than 180°, preferably more than 270°. Between the first and second end 55, 56 an opening 57 is formed. The intermediate portions are such that the second parts 5 extend, in top view, at least partly under and/or next to the first part 4, such that the first part 4 and second parts 5 have facing surfaces in close proximity. Close proximity is to be understood as partly against each other or distanced such that the distance between said facing surfaces is at least in part smaller than the thickness of the wall 11 to be clamped. The length W of the second parts 5, measured between the connecting intermediate parts 6 and the free ends 7 is preferably slightly greater than the size B of the first part, measured in the same direction, such that in top view the free ends 7 extend beyond the first part. This means that the wall 11 of a bodily part can at least be clamped in four positions between the first and second parts 4, 5.

An operation element 2 according to FIG. 6 can be used as follows.

A shunt 13 is prepared with a second ring 17 as shown in FIGS. 2C and D. Then the shunt 13 is extended through the central area 58 of the first part 4 of the operation element 2, defined by the curved portion 54. To this end the shunt 13 can be passed through the opening 57, elastically forcing the ends 55, 56 outward. Alternatively the shunt can be passed through said curved portion in a direction perpendicular to the plane P1. The shunt is positioned such that the folded wall part 24 thereof extends inside said curved portion 54 and the second ring 17 is moved against the first part 4, only the double, folded wall of the shunt 13 enclosed between said ring 17 and sad first part 4. Then the free ends 7 of the second parts 5 are forced into or through the wall 11 of the vessel 12 or such bodily part. By pushing the second parts 5 in or under said wall 11, the wall 11 and the second ring 17 within said folded end of the shunt 13 are clamped between said facing surfaces 4A, 5A of the first and second parts 4, 5, as shown in FIG. 7. This leads to a fluid tight connection. If necessary, sutures, staples or other means may be used to further secure the connection.

Alternatively the second parts 5 can be introduced into or through said wall 11 first and then forcing the second ring 17 with the double folded wall of the shunt 13 in between the first part 4 and the outside of the wall 11, through said opening 57.

FIG. 9 shows an even further embodiment of an operation element 2. In this embodiment the operation element is similar to the one as shown in FIG. 1. However, in this embodiment the first 4 and second part 5 are interconnected by an intermediate part 6 which is curved and encloses an angle of approximately 180°. Both the first and second parts 4, 5 are curved, preferably approximately circular or oval and enclosing an angle of more than 180°, more preferably more than 270°. In the embodiments shown at least the first part has an enclosed angle of at least approximately 360°, such that a central area is enclosed substantially fully by said first part 4. The first and second parts 4, 5 again are in parallel planes P1, P2 and have facing surfaces 4A, 5A which are in contact with each other or in close proximity, such that a wall 11 of a vessel 12 can be clamped in between.

In the embodiments shown the free end(s) of the or each second part 5 is/are shown sharp. However, they may be blunt. Puncturing means can then be provided by other means, for example a needle, scalpel or the like. Since the puncture is very small and immediately closed by the second part 5 and/or the intermediate part 6, blood loss or loss of other bodily fluids is no risk. The flat surface of the wall created in and by the operation element provides for a good connecting surfaces. Connections according to the present invention can be made both inside and outside a human or animal body, for example in training and testing. Since the first and second part are always connected to each other, no risk occurs that any part of the operation element or set will become detached inside a vessel or other bodily part. In the embodiments shown the or each second portion is shown extending under said wall, the intermediate portion extending through said wall. However, it is also possible to insert the free ends into the wall, between the outer surface and the inner surface thereof.

Shunts of any type, length or cross section can be used. The shunt 13 may be connected to the element 2 or part thereof, before or after connection of the element 2 to the wall 11.

The present invention is by no means limited to the embodiments of the present invention as have been shown by way of example. Many variants are possible within the scope of the present invention as defined by the claims.

A operation element may be of a different design and can have more then the number of windings shown. It could for example be part of a wound spring. In an embodiment similar to FIG. 3-9 the ring shaped element or at least the second part 5 could also be connected to the or a plug, such that the folded back end of the shunt and the wall of the vessel will be enclosed between the clamping parts of the ring shaped element, such as the lower wall end of the plug and the ring 41. The receiver would then lock the shunt against the plug 30. Other locking means for the connecting elements may be used, for example bayonet means, gluing means and the like. In theory the ring shaped element can have any form, dimension, size and shape. A substantially circular shape is favourable since it can then easily be rotated. An operation element, set or method according to the present invention can also be used in other areas of operation than vascular surgery, for example in bladder, bowels or the like but is especially suitable for by-pass surgery such as by-pass surgery in the brain.

What is claimed is:

1. An operation element for use in surgery, comprising a first part for positioning on the outside of a wall of a bodily part containing bodily fluid and at least a second part for positioning in or on the inside of said wall, said second part being connected to said first part and having a free end, for extending into and/or through said wall, wherein the first and second part have surfaces facing each other, such that during use a wall part of said wall is enclosed between said facing surfaces of said first and second part.

2. An operation element according to claim 1, wherein said first and second part extend substantially parallel to each other, such that during use a wall part enclosed between said first and second part is substantially flat.

3. An operation element according to claim 1, wherein said first part has a curved configuration enclosing at least a circle segment of more than 180 degrees.

4. An operation element according to claim 1, wherein said operation element comprises or is formed by a ring shaped element having windings, preferably at least one-and-a-half winding, wherein said first part comprises part of said windings and said second part comprises a further part of said windings, wherein said second part is preferably provided with at least one sharpened end for forming puncturing means.

5. An operation element according to claim 4, comprising more than one-and-a-half winding but less than two windings.

6. An operation element according to claim 4, wherein said ring shaped element comprises a central axis and defines a substantially cylindrical outer surface, wherein said sharpened end is bent such that it lies substantially in or parallel to said outer surface.

7. An operation element according to claim 6, wherein said sharpened end is bent back into the direction of winding.

8. An operation element according to claim 4, wherein the ring shaped element comprises said first part and said second part, the first and second part being substantially flat and being connected to each other by a connecting part, such that the first and second part lie substantially parallel to each other.

9. An operation element according to claim 1, wherein said first part comprises a curved segment including an angle of more than 180 degrees, having opposite first and second ends, a second part connected to at least one of said first and second ends by a curved intermediate part, wherein said at least one second part at least partly extends in a plane substantially parallel to a plane defined by at least said first and second ends of said first part and at least one further part of said first part between said first and second ends, which second part has a free end opposite said intermediate part which is spaced apart from said first and second ends.

10. An operation element according to claim 9, wherein said second part includes a curved segment with a direction of curvature which is comparable to the direction of curvature of said curved segment of said first part.

11. An operation element according to claim 9, wherein a said second part is connected to each of said first and second end by an intermediate part, which second parts at least partly extend in a plane and preferably define a plane, substantially parallel to a plane defined by at least said first and second ends of said first part and at least one further part of said first part between said first and second ends.

12. An operation element according to claim 11, wherein said second parts comprise each a substantially straight portion, extending from said intermediate part, said substantially straight portions extending substantially parallel to each other, in a view perpendicular to said plane partly under and/or next to said curved segment of said first part.

13. An operation element according to claim 11, wherein each of said second parts comprises a sharpened end for forming a puncturing means.

14. An operation element according to claim 1, wherein said first and second part are at least partly of wire material in close proximity.

15. An operation element according to claim 14, wherein the wire has a substantially circular cross section or a cross section with a flattened portion, such that of the ring shaped elements touching parts are said flattened portions.

16. An operation element according to claim 15, wherein the wire has a diameter of less than 2 mm.

17. An operation element according to claim 4, wherein said ring shaped element is connected to or part of a tube shaped element.

18. An operation element according to claim 9, wherein said first part is connected to or part of a tube shaped element.

19. An operation set for connecting a shunt to a bodily part comprising an operation element according to claim 1 and an element for connection to said operation element.

20. An operation set according to claim 19, wherein said connecting element comprises at least two hook shaped elements, designed for hooking around a first part of said operation element, preferably around at least one winding thereof.

21. An operation set according to claim 19, wherein said connecting element comprises a tube having a central canal, having a cross section approximately equal to the cross section of said ring shaped element.

22. An operation set according to claim 19, wherein the set further comprises a second ring having a central opening, preferably having a cross section substantially equal to the cross section of said first part of said operation element.

23. An operation set according to claim 19, wherein furthermore a clamping element is provided.

24. An operation set according to claim 22, wherein said first part comprises a curved segment including an angle of more than 180 degrees, having opposite first and second ends, an opening being defined between said first and second ends which has a width between said first and second ends which is smaller than the diameter of said cross section of said second ring, and a second part connected to at least one of said first and second ends by a curved intermediate part, wherein said at least one second part at least partly extends in a plane substantially parallel to a plane defined by at least said first and second ends of said first part and at least one further part of said first part between said first and second ends, which second part has a free end opposite said intermediate part which is spaced apart from said first and second ends.

25. An operation set according to claim 19, wherein furthermore an instrument is provided for engaging said ring shaped element and turning said ring shaped element over at least 270°

26. A set of an operation set according to claim 19 and a shunt such as an artificial shunt or a blood vessel.

27. A set according to claim 26, wherein said shunt has an internal cross section substantially equal to the cross section defined by said curved segment of said first part of said operation element of the operation set.

28. A set according to claim 26, wherein said shunt has a first end which is folded back over a ring, preferably said second ring.

29. A set of an operation set according to claim 24, wherein said shunt has a diameter near said ring which is larger that de width between said first and second end of said curved segment of said first part of the operation element.

30. A method for connecting a shunt to a bodily part such as a blood vessel, for example an artery or vein, comprising the steps of:
inserting at least one supporting second part of an operation element into and/or through the wall of a bodily part;
positioning said second part within said bodily part against or in said wall and positioning a first part of said operation element, which is connected to said second part, against the outside of said wall, thereby forcing a part of said wall enclosed between said first part and said second part in a substantially planar form; and
connecting a shunt to said operation element.

31. A method according to claim 31, using a substantially ring shaped element, comprising the steps of:
sticking a front end of said ring shaped element through said wall, said ring shaped element having more than one winding;
moving said ring shaped element such that at least a second part of at least one winding is brought in or under said wall, whereas a first part of the ring shaped element is kept on the opposite outer side of said wall;
whereby said part of said wall is clamped between said part of said winding under said wall and said part of said ring shaped element on the outer side of said wall.

32. A method according to claim 31, wherein a ring shape element is used having a first winding and a second winding, connected by a connecting part, whereby said first winding is positioned under said wall and said second winding is positioned on said outside of said wall, the connecting part extending through at least part of said wall.

33. A method according to claim 31, wherein the ring shaped element is rotated around a longitudinal axis for bringing said part of said at least one winding under said wall.

34. A method according to claim 30, using an operation element of which said at least one second part is connected to a first or second end of a curved segment of the first part, by an intermediate part, said second part comprising a curved segment extending alongside at least part of said curved segment of said first part.

35. A method according to claim 30, using an operation element comprising two second parts, respectively connected to a first and second end of a curved segment of the first part, by an intermediate part, said second parts extending at least partly in a plane substantially parallel to a plane defined by said first and second end and a further part of said curved segment of said first part, wherein the second parts are positioned in or under said wall.

36. A method according to claim 30, wherein a shunt is connected to said first part of the ring shaped element on the outside of said wall.

37. A method according to claim 36, wherein a shunt is used having a substantially tube like form, wherein a first end of said shunt is folded back over a ring, such that said ring is enclosed by said shunt, forming a folded edge supported by said ring, whereby said folded edge is positioned against said first part on the outside of said wall and is connected to said operation element and/or said wall in a fluid tight position.

38. A method according to claim 36, wherein a clamping element is moved over the outside of said shunt for clamping said folded edge to said operation element.

39. A method according to claim 29, wherein a shunt is connected to said ring shaped element, after which part of said wall extending within a central opening of said first part of said operation element is removed.

40. A method according to claim 39, wherein said wall part extending within said central opening is sucked into said central opening forming a convex surface, before removing of said part of said wall.

41. A bodily part, provided with an operation element having a first and at least one second part, connected to said first part by an intermediate part, said first part extending outside said bodily part and said at least one second part extending inside said bodily part, said intermediate part extending into or through the wall of said bodily part, said first and second part enclosing and clamping a wall part of said bodily part, said wall part being substantially flattened by said first and said at least one second part.

42. A bodily part according to claim 41, wherein said first part comprises a curved portion, a shunt being connected to said operation element such that a fluid tight connection is provided between an inner portion of said bodily part and an inner portion of said shunt, said fluid connection extending through a central portion of said first part of said operation element.

43. A bodily part according to claim 41, wherein said shunt has a free end folded back over a second ring such that said shunt has, near said second ring, a diameter which is larger than the width of an opening defined between said first and second end of said curved segment of said first part, whereby said second ring is forced between said curved segment of said first part and said wall part, said shunt extending through said first part.

44. A bodily part according to claim 43, wherein said first part is elastically deformable, such that said second ring can be forced between said first part and said wall part by forcing said free end through said opening between said first and second end.

45. A method for preparation of an operation set for vascular surgery, comprising the steps of, providing an operation element according to claim 1, a ring and a shunt, said operation element having a central area substantially equal to a central opening of said ring and a width in at least one direction substantially equal to or smaller than the diameter of the bodily part to be used.

46. An operation element for use in surgery, comprising a first part for positioning outside on the outer wall of a bodily part containing a bodily fluid, at least a second part for positioning inside said vessel, in or in contact with said wall, said second part being connected to said first part by an intermediate part, said first and second parts have surfaces facing each other wherein, during use said surfaces enclose said wall from opposite sides, directly or by means of pressure on other parts and, the surfaces directly in contact with the wall exerting pressure on the wall from opposite sides are arranged to define essentially parallel planes and all parts arranged not to obstruct a central, essentially cylindrical lumen of a shunt to be connected to said first part, the axis of which at said first part encloses an angle of less than 60 degrees with the normal axis of both of the planes created by said facing surfaces of said first and second parts enclosing said vessel wall.

47. An operation element according to claim 15, wherein the wire has a diameter of less than 1 mm.

48. An operation element according to claim 15, wherein the wire has a diameter of approximately 0.25 mm.

49. A set according to claim 28, wherein the ring is said second ring.

50. A method according to claim 39, wherein said operation element is removed by laser.

51. An operation element for use in surgery according to claim 46, wherein said first part encloses an angle of less than 45 degrees.

52. An operation element for use in surgery according to claim 46, wherein said first part encloses an angle of about 0 degrees.

53. An operation set according to claim 19, wherein said bodily part is a vein or an artery.

54. An operation element for use in surgery according to claim 1, wherein said surgery is vascular surgery.
